(19) United States
(12) Patent Application Publication
(10) Pub. No.: US 2015/0305932 A1
(43) Pub. Date: Oct. 29, 2015

(54) Intraocular Injection Guide

(71) Applicant: Vikas Rammohan Maturi, Carmel, IN (US)

(72) Inventor: Vikas Rammohan Maturi, Carmel, IN (US)

(21) Appl. No.: 14/262,832

(22) Filed: Apr. 28, 2014

(51) Int. Cl. A61F 9/00; A61M 5/46

(52) U.S. Cl. CPC A61F 9/0026; A61M 5/46

(57) ABSTRACT

An intraocular device to assist a physician for injecting into an eyeball by aligning a device to aspects of the eyeball. The device may be adapted to control the depth of an injection needle and to align the correct placement of the needle.

### FIELD

The invention relates to an intraocular injection guide that assists a health care professional with intraocular injections.

### BACKGROUND

Intraocular injections are necessary to treat various ocular conditions. For example certain anti-VEGF injections are used to treat very common diseases such as age related macular degeneration (AMD) and diabetic retinopathy. In this treatment, the surgeon will need to hold the eyelids open by using a speculum. The speculum pries the eyelids open to preven the eyelids from snapping back (such as blinking) especially during the critical portion of the injection, and thus prevents an infection from developing (as the lids contain a large number of bacteria).

After prying open the eyelids, the surgeon then bathes the entire eye area with topical anesthetic and then with antisepetic solution. The problem with the current standard of care is that the speculum used to hold the eye open is generally considered quite painful. Many patients rate the pain of the speculum as greater than the pain of the needle penetrating the eye. This is because anesthetic drops used for the procedure numb the eyeball but are not as effective on the skin of the eyelid. Additionally, the current method leaves the eye pried open for almost a minute, drying the surface of the eye (the cornea) which leads to discomfort after the procedure and sometimes leads to a corneal abrasion (or scratch). The third disadvantage with the current method is that topical antiseptic is applied to the entire surface of the eye multiple times, which can be wasteful and cause discomfort for approximately 24 hours after the procedure.

For injections, the physician will then inject by inserting the needle through the sclera such that the needle tip will be in the viterous body—the central cavity of the eye, filled with vitreous humor. The physician then depresses the needle plunger and the medicine is injected into the vitreous body.

The problem that happens that given small geography to work with and that the eyelids are autonomically programmed to blink, the physician has to be very precise with the injection and work quickly to inject so that the speculum can be removed and the eyelids can return to autonomic blinking. The small geography also requires the physician to be precise in injecting the needle into the sclera at a precise location so as to avoid the lens, the iris, and the ciliary muscles. Secondly, the surgeon must visually confirm that the needle is perpendicular to the sclera at all times to avoid damaging the retina or lens (due to improper angulation). In addition, the physican must be precise in the depth of the needle penetration into the viterous body.

Other devices exist that assist physicians with guiding the needle into the eye. Those devices, though, suffer from certain infirmities. First, many still require the use of a speculum to prop the eyelids open. This causes patient discomfort. Other devices are also not shaped appropriately to conform to the curvatures of the eye; that is gaps exist between the device eye surface and the eye surface itself. The means that an eyelid can close under the device, thereby pushing the device up away from the eye surface which can lead to potential contamination of the needle tip. Other devices are also not interchangeable with the various size syringes or needles. Because different medicines are used in filled syringes, the sizes of the syringe body, syringe tip, and gauge size of the needles all vary and as such, the intraocular devices in the art do not generally allow for interchangeability.

### SUMMARY

The present invention is directed to an introcular injection device that succeeds where many other devices fail. The device may include an intraocular injection device that permits the physician to grasp the device, align the device effectively with the cornea to ensure precise and replicable location, and allow interchangeability of the device with various syringes.

### BRIEF DESCRIPTION OF THE DRAWINGS

For the purpose of facilitating an understanding of the subject matter sought to be protected, there is illustrated in the accompanying drawing embodiments thereof, from an inspection of which, when considered in connection with the following description, the subject matter sought to be protected, its construction and operation, and many of its advantages should be readily understood and appreciated.

Various drawings are included to show non-limiting features of embodiments of the invention.

FIG. 1 is a cross sectional view of an embodiment of the invention.

FIG. 2 is a view of one surface of an embodiment of the invention.

FIG. 3 shows another view of a surface of an embodiment of the invention.

FIG. 4 represents an alternative cross sectional view of an embodiment of the invention.

FIG. 5 represents another cross sectional view of another embodiment of the invention.

FIG. 6 represents another view of an embodiment of the invention. FIG. 6 represents another cross sectional view of another embodiment of the invention.

FIG. 8 demonstrates an exploded view of another embodiment of the invention.

FIG. 9 represents an embodiment of the invention in operation.

### DETAILED DESCRIPTION

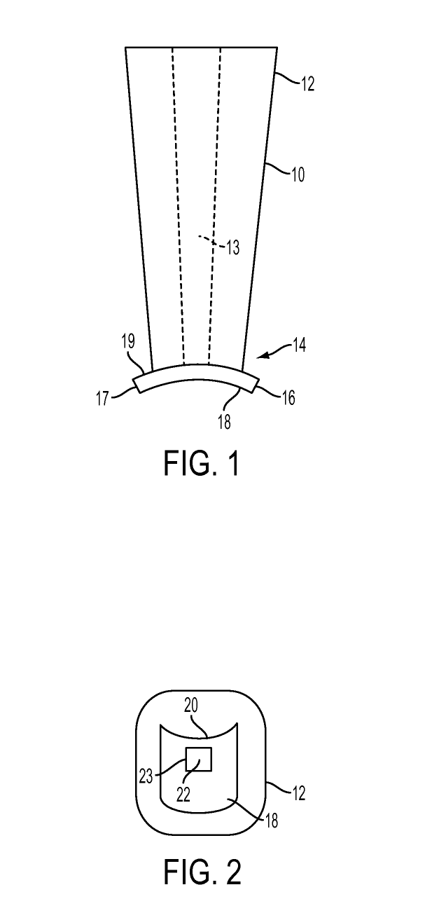

FIG. 1 represents an outer view of the intraocular injection device 10. The device 10 includes an injector receiving end 12 and a corneal end 14. The device in whole or in part may be cylindrical, rectangular, or otherwise shaped. Extending from the injector receiving end 12 and the corneal end 14 is a guiding channel 13, which is akin to a lumen connecting the two ends. The guiding channel 13 shape may be cylindrical or edged. The width or diameter of the guiding channel 13 is adapted to receive an injection device. Disposed near the corneal end 14 is an end flange 16, which has an end flange outer surface 17 that abuts the corneo-scleral junction, a scleral surface 18, and an end flange top surface 19.

The scleral surface 18 is curved in such a way as to sit on the eye surface and create a good fit with the eye surface. The cornea is slightly oval, having an average diameter of about 12 mm horizontally and 11 mm vertically. Accordingly, but not limited, the scleral surface 18 may have a curvature that would create an 24 mm diameter. Said another way, the scleral surface 18 may be adapted to match the curvature of the eye surface. The scleral surface 18 may be made of a material that is resilient as to conform to the eye surface irrespective of its curvature, semi-resilient for the same reason, or rigid. Exemplary materials of the device and/or flange may be made of medical device grade materials. The end flange 16 may be made uniformly with the corneal end 14 or may be separately made and affixed to the corneal end 14 thereafter. The end flange outer surface 17 may be tapered, flush edged, or otherwise shaped as to allow an eyelid that comes in contact with the end flange outer surface 17 to stop at the edge or to slide over the end flange top surface 19. Similarly, the junction (if any) of the end flange top surface 19 and the end flange outer surface 17 may be rounded or tapered to facilitate eyelid movement over the the top surface 19.

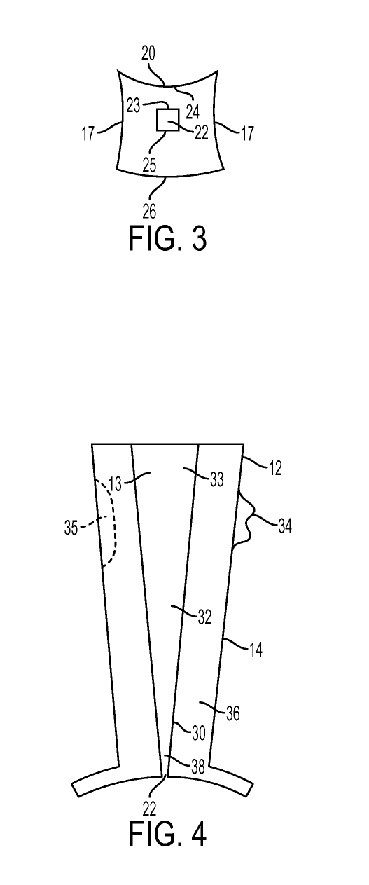

FIGS. 2 and 3 represent a surface view of the introcular injection guide 10 by showing the scleral surface 18. Along one side of the device 10 is a corneal edge 20. The corneal edge 20 is concave shaped forming a rounded edge. Corneal edge 20 may also be curved in such as manner as to follow the curvature of the cornea of an eye. Disposed through the scleral surface in fluid communication with the guiding channel 13 is a needle exit 22. Needle exit 22 is configured to allow the needle of the injector to exit the device 10 and allow for injection into the eye. Along scleral surface 18, the needle exit 22 is configured to be closer to the corneal edge 20. More particularly the needle exit 22 has a needle exit corneal edge 23 that is closest to the corneal edge 20. In different embodiments, the needle exit corneal edge 23 is between 3 mm to 5 mm from the corneal edge 20 and more particularly can be at any distance therebetween.

FIG. 3 also shows a view of scleral surface 18. Corneal edge 20 may also include a corneal edge center 24 that can be the center of the corneal edge 20 curvature. Needle exit 22 also includes a needle exit opposite side 25 that is opposite to the needle exit corneal edge 23. As such the needle exit 22 may be configured so as to have the needle exit corneal edge 23 be 3-4 mm from the corneal edge 20 with the needle exit opposite side 25 being 4-5 mm from the corneal edge 20. In another embodiment, the needle exit corneal edge 23 may be 3 mm from the corneal edge center 24 and the needle exit opposite side 25 may be 5 mm from the corneal edge center. In this regard, the configuration may define at least one dimension for the size of the needle exit 22. If needle exit 22 is not square or rectangular or is round or otherwise shaped, needle exit corneal edge 23 is a part of the needle exit that is closest to the corneal edge and where needle exit opposite side 25 is a part of the needle exit farther away from needle exit corneal edge 23. In another embodiment, the scleral surface 18 may include a corneal edge opposite side 26 such that the needle exit 22 is closer to the corneal edge 20 than to the corneal edge opposite side 26.

FIG. 4 shows another view of the device 10, in cross section. The guiding channel 13 includes a lower tapered end 30 that terminates near the corneal end 14. The lower tapered end 30 also is in communication with the lower lumen 32 and upper lumen 33 of the guiding channel 13. The device 10 may include one or more finger grips 34, 35, which may be indented or protruding from the surface of device 10. The lower lumen 32 within the guiding channel 13 may include a syringe blocker 36, which may be integrally made with the lower tapered end 30 or lower lumen 32, or may be separately made and inserted into the lower lumen. The syringe blocker 36 may be shaped to block a syringe inserted into the lumen, thereby controlling the penetration depth of a syringe and/or a needle. The syringe blocker 36 may be a bulge, protrusion, bump, or anything that may block the penetration depth of a syringe. At the end of the lower tapered end 30 may include a needle guide 38, which is configured to ensure that a needle of an injection device can slide through the guide 38 easily and into the needle exit 22. The needle guide 38 may also be configured so that only a certain length of the needle may exit out of the needle exit 22 and thus only a certain length of needle penetrates the eye. The needle guide 38 may work in conjunction with, or independent of, syringe blocker 26.

FIG. 4 also shows the end flange 16, the end flange 16 has the end flange outer surface 17 and end flange top surface 19. The end flange 16 also has a junction 40 where the flange 16 is connected to the corneal end 14. The junction 40 can be an abrupt or defined boundary or junction or may be a more tapered or non-defined boundary. Notwithstanding, a distance may be defined by the junction 40 extending to the end flange outer surface 17. In a non-limiting embodiment, the distance may be between 1-3 mm. The end flange 16 may be disposed around three sides of the corneal end 14, with the fourth side being the corneal edge 20.

Also shown in FIG. 4, the upper lumen 33 is shown having a wider opening than the lower lumen 32. The progressive narrowing can narrow from a wider opening 10-13 mm to a narrower width of 4-5 mm. It should be understood that these dimensions are exemplary only.

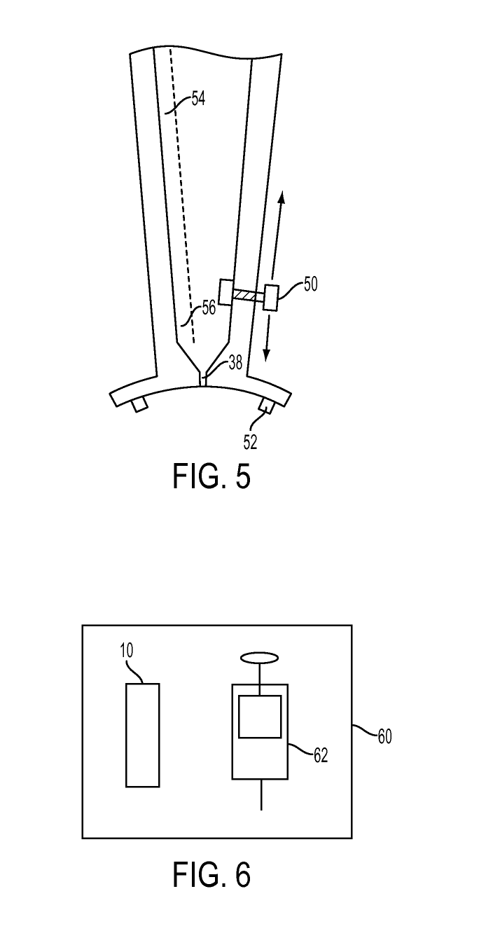

FIG. 5 shows another embodiment. In this Figure, the device 10 may include a moveable syringe blocker 50 that can be height adjusted up or down the device 10 such that the depth of an inserted syringe can be controlled. Another independent feature includes a scleral positioner 52 that helps hold or secure the device to the sclera. For example, scleral positioner 52 may be rounded bumps on the scleral surface 18 that are sufficient to depress onto the sclera and assist the practitioner in aligning the device with the cornea and maintaining such position. The scleral surface 18 may include a single or a plurality of positioners 52. The positioner 52 may be integrally made with, or made separately from, the scleral surface. In yet another embodiment (not shown), the scleral surface 18 may include indents or holes into which one or more positioners 52 may be inserted.

FIG. 5 also shows another independent feature that includes an inner tube 54 that extends through the guiding channel 13. The inner tube 54 terminates at an inner tube exit 56, which is adapted to allow for fluid communication between the lumen of the inner tube 54 and the lower lumen 32 and/or needle guide 38. In function, the user can pour liquid into inner tube 54 and have the contents exit into the lower lumen 32 or guide 38 and subsequently into needle exit 22. In this embodiment, fluid entering the inner tube 54 will not generally interfere with the syringe insertion into the device.

FIG. 6 demonstrates another alternate embodiment in which the device 10 is packaged with a syringe device in a kit 60. The syringe 62 may be a prefilled syringe or may comprise an empty syringe and vial of the medicine. The kit 60 may include other parts, including but not limited to, antiseptics, anesthetics, or device inserts. Exemplary, not limiting, syringes may include medicines to treat age related macular degeneration, intraocular pressure, diabetic retinopathy, or infection. Without limitation, such medicines may include suitable agents that can be selected from, for example, small molecules, such as steroids and NSAIDs, proteins, enzymes, hormones, oligonucleotides, polynucleotides, nucleoproteins, modified DNA and RNA loaded viruses with modified capsid, polysaccharides, glycoproteins, lipoproteins, polypeptides, including drug carriers, such as pokymers, micro and nano particles.

Further examples of agents useful in this invention include, without limitation, atropine, aflibercept, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, bevacizumab, and other antibodies, antineoplastics, anti-VEGFs, ciliary neurotrophic factor, brain-derived neurotrophic factor, bFGF, caspase-1 inhibitors, caspase-3 inhibitors, α-Adrenoceptors agonists, NMDA antagonists, Glial cell line-derived neurotrophic factors (GDNF), pigment epithelium-derived factor (PEDF), NT-3, NT-4, NGF, IGF-2, antibiotics or antifungal drugs, anti-pain medication, anesthetics, and combinations thereof.

FIG. 7 demonstrates an alternate embodiment of the device 10. In this embodiment, there is an outer housing 70 that can be integrally made with or separately made from the end flange 16. The outer housing 70 may include one or more of the features shown in any other embodiment. The outer housing 70 also includes an outer housing inner surface 72 and a housing lumen 73. This embodiment demonstrates an insertable insert 74 into the lumen 73. The insert 74 has an insert outer wall 76 that is disposed to face the outer housing inner wall 72 at one or more points along the inner wall 72. The insert outer wall 76 is adapated to fit into the outer housing 70. Within the insert 74 near its insert bottom 75 may include a syringe stopper 77 and an insert needle guide 78. The insert needle guide 78 is adapted to be aligned with the needle exit 22 of the end flange 16. The syringe stopper 77 is adapted to control the depth of the needle penetration into the eye. In this regard, different inserts 74 can be used in the same outer housing 70 and upon use, the inserts 74 can be swapped out. Furthermore, with different size syringes, different inserts 74 can be used wherein the insert chosen controls the depth of the needle into the eyeball.

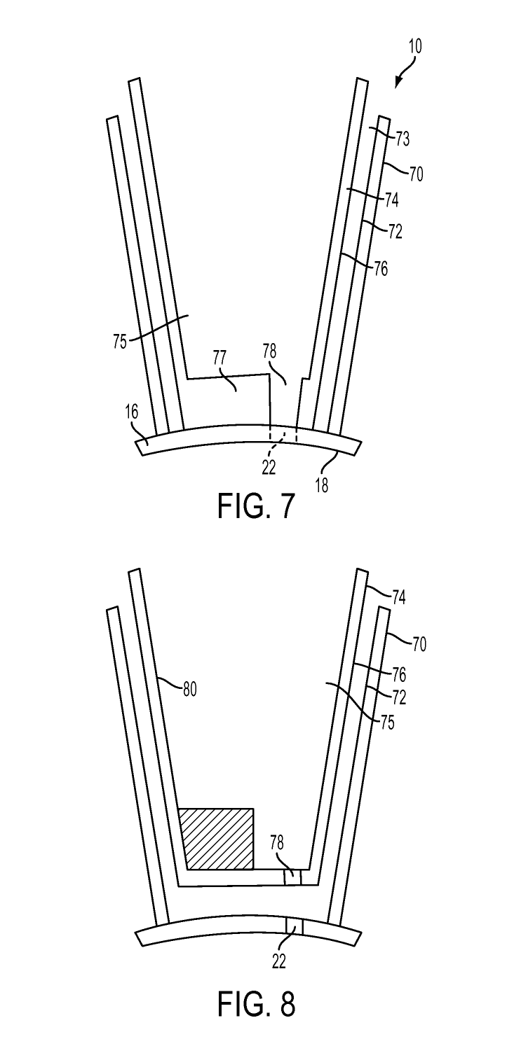

FIG. 8 demonstrates the alternate embodiment of FIG. 7 in an exploded view, showing the insert 74 insertable into, but not inserted into, the outer housing 70. Though FIGS. 7 and 8 demonstrate that the outer housing inner wall 72 and insert outer wall 76 are angled, they need not be so. Either wall may be straight or angled. Similarly, insert inner wall 80 may be straight or angled.

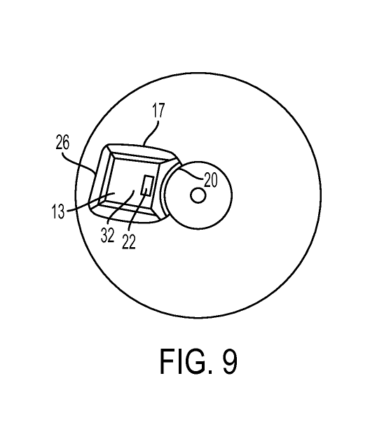

FIG. 9 demonstrates the device 10 in use by looking “down” the device from the injector receiving end 12 along the length of the device 10 to the corneal end 14. The user places the device 10 onto the surface of the eye. The user aligns corneal edge 20 along the boundary of the cornea. The user can hold the device 10 so that the user's fingers are holding the device along the same sides as end flange outer surface 17, with in certain embodiments, holding the device 10 using finger grips 35.

In this FIG. 9, as the device 10 is placed on the eye and corneal edge 20, the user can insert the syringe into the device 10 and, depending on the type of syringe blocker used, can then guide the syringe needle into the various needle guides and into the needle exit for injection into the eye. Because of the positioning of the needle exit 22, the syringe needle will inject the eye at a proper and controlled location. In addition, the syringe blockers, when used, can control the depth of the injection into the eye. Also, as the device 10 is placed on the eye, the eyelids are permitted to move (because no speculum is needed to pry the eyelids open during the injection), the eyelids will contact the end flange outer surface 17 and are permitted to ride over the end flange surface 19. In this regard, even if the eyelids do close, which makes the procedure and comfortable to both the user and the patient, the eyelids are out of the way of the injection site.

When the device 10 is properly placed, the user then can deposit some antiseptic into the device, which ultimately will exit the needle exti 22 and cleanse the area to be injected. In this regard, by using the device 10, only a certain area of the eye needs to be cleansed whereas with other devices, the entire eye will be cleansed. The user then can insert the syringe into the device 10 and depending on the form or feature of the syringe stopper, the user then can inject the eye, thereby maintaining proper location and depth of the injection.

As used herein, the term “device equivalent” means any subsequent device that is approvable by a governmental health regulatory agency wherein that device is deemed equivalent to this device 10, under health regulatory regulations. For example, but not limited to, under current U.S. FDA guidelines, a later device may be deemed substantially equivalent to this device 10 as a so-called 510(k) clearance. If for example the device 10 is deemed to be a predicate device, then a subsequent device is a substantial equivalent if in in comparison to a predicate, the subsequent device: (A)(i) has the same intended use as the predicate; and (ii) has the same technological characteristics as the predicate; or (B)(i) has the same intended use as the predicate; and (ii) has different technological characteristics and the information submitted to FDA; and (a) does not raise new questions of safety and effectiveness; and (b) demonstrates that the device is at least as safe and effective as the legally marketed device.

The manner set forth in the foregoing description and accompanying drawings and examples, is offered by way of illustration only and not as a limitation. More particular embodiments have been shown and described, and it will be apparent to those skilled in the art that changes and modifications may be made without departing from the broader aspects of the disclosure. The actual scope of the protection sought is intended to be defined in the following claims when viewed in their proper prospective based on the prior art.

What is claimed is:

1. An intraocular injection device, comprising:
(a) an elongated tubular member having an injector receiving end and a scleral end, with the scleral end further comprising an end flange disposed at the corneal end;
(b) a guiding channel extending from an injector receiving end to the end flange, the guiding channel having a lower lumenal end and a lower lumenal end, the lower lumenal end further having a syringe stopper, the syringe stopper is adapted to control the depth of a needle penetration into an eyeball;
(c) the end flange being disposed around three sides of the corneal end, the end flange also including a scleral surface disposed on one side of the end flange;
(d) the scleral surface is curved to match the corneal or scleral surface of an eyeball, the surface further having a corneal edge that is adapted to align to a cornea of the eyeball; and
(e) the end flange further including a needle exit, the needle exit having a needle exit corneal edge that is no less than 3 mm from the flange corneal edge.

2. The intraocular injection device of claim 1, in which the lower lumenal end includes a syringe stopper.

3. The intraocular injection device of claim 2, in which the syringe stopper is adapted to block a syringe inserted within the guiding channel.

4. The intraocular injection device of claim 2, in which the syringe stopper is adapted to control the depth of a needle penetration into an eyeball.

5. The intraocular injection device of claim 1, in which the end flange is also disposed around three sides of the corneal end.

6. The intraocular injection device of claim 4, in which the scleral surface is curved to match the corneal or scleral surface of an eyeball.

7. The intraocular injection device of claim 1, in which the scleral surface has a corneal edge, the corneal edge adapted to align to a cornea of the eyeball.

8. The intraocular injection device of claim 1, in which the needle exit has a needle exit corneal edge that is more than 3 millimeters but no more than 4 millimeters from the corneal edge.

9. The intraocular injection device of claim 1, in which the needle exit has a needle exit corneal edge that is more than 4 millimeters but no more than 5 millimeters from the corneal edge.

10. The intraocular injection device of claim 1, in which the needle exit has a needle exit corneal edge that is more than 5 millimeters but no more than 6 millimeters from the corneal edge.

11. The intraocular injection device of claim 1, in which the needle exit has a needle exit corneal edge that is more than 6 millimeters but no more than 7 millimeters from the corneal edge.

12. The intraocular injection device of claim 1, in which the device is a device equivalent.

13. The intraocular injection device of claim 1, in which the guiding channel includes a lower tapered end, the lower tapered end terminating near the lower channel end.

14. The intraocular injection device of claim 13, in which the lower tapered end is in communication with the upper channel end and the lower channel end.

15. The intraocular injection device of claim 1, in which the elongated tubular member includes one or more finger grips, the finger grips indented or protruding from the surface of the elongated tubular member.

16. An intraocular injection device, comprising:
(a) an elongated tubular member having an injector receiving end and a scleral end, with the scleral end further comprising an end flange disposed at the corneal end;
(b) a lumen extending from an injector receiving end to the end flange, the lumen having a lower lumenal end and a lower lumenal end, the lower lumenal end further having a syringe stopper, the syringe stopper is adapted to control the depth of a needle penetration into an eyeball;
(c) the end flange being disposed around three sides of the corneal end, the end flange also including a scleral surface disposed on one side of the end flange;
(d) the scleral surface is curved to match the corneal or scleral surface of an eyeball, the surface further having a corneal edge that is adapted to align to a cornea of the eyeball; and
(e) the end flange further including a needle exit, the needle exit having a needle exit corneal edge that is no less than 3 mm from the flange corneal edge;
(f) the end flange also including at least one end flange surfaces adapted to permit an eyelid to smoothly travel over the at least one end flange surface.

17. The intraocular device of claim 16, wherein the scleral surface further includes at least one scleral surface positioner adapt to help hold the device onto a sclera or cornea of the eyeball.

18. The intraocular device of claim 16, wherein the elongated tubular further includes an insert that is adapted to fit inside in the lumen.

19. The intraocular device of claim 16, wherein the device includes at least one of: (a) at least one finger grip on a surface of the device that is not on the same surface as the corneal edge; (b) an insertable insert adapted to be placed into the device lumen; (c) one or more scleral positioners adapted to hold the device on to a sclera or cornea of the eyeball; (d) a moveable syringe stopper adapted to control the depth of a needle; and (e) any combination of (a) to (d).

20. An intraocular injection device kit, the kit comprising:
(a) an elongated tubular member having an injector receiving end and a scleral end, with the scleral end further comprising an end flange disposed at the corneal end;
(b) a lumen extending from an injector receiving end to the end flange, the lumen having a lower lumenal end and a lower lumenal end, the lower lumenal end further having a syringe stopper, the syringe stopper is adapted to control the depth of a needle penetration into an eyeball;
(c) the end flange being disposed around three sides of the corneal end, the end flange also including a scleral surface disposed on one side of the end flange;
(d) the scleral surface is curved to match the corneal or scleral surface of an eyeball, the surface further having a corneal edge that is adapted to align to a cornea of the eyeball;
(e) the end flange further including a needle exit, the needle exit having a needle exit corneal edge that is no less than 3 mm from the flange corneal edge; and
(f) a medicine either in a vial or in a prefilled syringe, the medicine comprising at least one of an anti-VEGF, a prostaglandin, a rapamycin or derivative thereof, an antiretroviral, a peptide or polypeptide, a steroid, a NSAID, a carbonic anhydrase inhibitor, a beta-andrenergic receptor antagonist, and an immunosuppressant; or combination thereof.
